(19) United States
(12) Patent Application Publication
(10) Pub. No.: US 2014/0193521 A1
(43) Pub. Date: Jul. 10, 2014

(54) Drug Delivery Systems and Method of Reducing Foal Infection by Treating Mares with Gallium Salts

(71) Applicant: Benjamin R. Buchanan, Navasota, TX (US)

(72) Inventor: Benjamin R. Buchanan, Navasota, TX (US)

(21) Appl. No.: 14/238,579

(22) PCT Filed: Aug. 10, 2012
(86) PCT No.: PCT/US2012/050430

§ 371 Date: Feb. 12, 2014

Related U.S. Application Data
(60) Provisional application No. 61/523,579, filed on Aug. 15, 2011.

(51) Int. Cl. A61K 33/24

(52) U.S. Cl. CPC A61K 33/24
USPC 424/650

(57) ABSTRACT

This invention relates to drug delivery systems and methods of preventing respiratory bacterial infection in a foal of a mare. This invention relates to the reduction of transmission from the mare to the foal of bacteria though contaminated matter in the foal's environment.

### BACKGROUND OF THE INVENTION

1. Field of the Invention

This invention relates to the veterinary arts. In particular it relates to newly discovered drug delivery systems and methods of preventing respiratory bacterial infection in a foal of a mare.

2. Description of the Related Art

Peripartum mares shed many pathogenic bacteria in their feces. Depending on the husbandry of the environment, there is a potential for the spread of these pathogens to the neonatal foals. Example of such pathogenic bacteria are Rhodococcus equi (“R. equi”).

Bacterial Pathogens

A major cause of foal pneumonia is a facultative gram-positive coccoid bacterium found in soils around the world, Rhodococcus equi (R. equi). Only the isolates containing virulence associated plasmids (“vap”), and especially vapA, are thought to cause clinical disease.

Foals are infected shortly after birth from environmental contamination with R. equi. Evidence points to the immature or ineffective innate immune response as a most likely reason for infection very early in life.. One hypothesis for the etiology of foal pneumonia is that the foal is infected shortly after birth and the disease slowly develops over the next 3-12 weeks. Prior to the introduction of antibiotics, fatality rates from R. equi could approach 80%. Even with improved antibiotics and monitoring of foals, fatality rates have been reported between 10% and 20%. The foals that survive the disease have a decreased chance of racing as an adult, indicating that the pneumonia may have long term effect as well as limit the athletic potential of the horse.

No vaccination strategy of the mare or the foal has shown any effect on limiting the development of R. equi pneumonia. Current clinical practices focus on trying to boost the foals immune system with doses of intravenous plasma at birth and again at 30 days. Other chemo prophylactic measures are currently being used including the administration of antibiotics from birth to 10 days of age. In addition to trying to prevent the disease, many farms have started to serially screen their foals with thoracic ultrasound in hopes of identifying the disease early and shortening the duration of therapy. The increased use of antibiotics applies selective pressure on the bacterium and antibiotic resistance is being reported in up to 5% of isolates.

It was initially believed that the soil was the source of the infectious bacteria, but recent studies do not show any association with isolates or concentrations in the soil and the incidence of pneumonia attributable to R. equi. However, virulent R. equi were commonly recovered from soil of horse breeding farms.

An association between R. equi pneumonia and the airborne burden of R. equi has been shown on farms in Australia. Further, the odds of detecting airborne R. equi was 173 times greater in stalls than in paddocks. Concentrations in the air, but not in the soil, are important in the development of R. equi pneumonia and the infection of foals early in life. When mares were evaluated as a potential source of environmental contamination, it has been shown that every peripartum mare tested sheds R. equi at time points in the week before and after foaling. Safe treatment of peripartum mares could lead to a reduction in the level of R. equi, shed in the feces. This may improve air quality and lower the incidence of R. equi pneumonia on the farm. Unfortunately, the drugs currently used for treating R. equi in foals cannot be safely used in the pregnant mare.

Gallium

Gallium (symbol Ga & atomic number 31) is a silvery soft metal that not exist in free form in nature. It is extracted as a trace component in bauxite, coal, diaspore, germanite, and sphalerite. Gallium salts, such as gallium citrate and gallium nitrate, are used as radiopharmaceutical agents in nuclear medicine imaging. The free dissolved gallium ion Ga³⁺ is the active component. For such, a radioactive isotope such as ⁶⁷Ga is used. Gallium competes for uptake with iron into iron binding proteins, and thus it concentrates in areas of inflammation, such as focal sites of infection, and also areas of rapid cell growth. Gallium is useful in the demonstrating the presence of malignant tissue. Gallium maltolate is being investigated in clinical and preclinical trials as a potential treatment for cancer, infectious disease, and inflammatory disease, because of its antiproliferative activity. These studies take advantage of gallium's similarity to iron, and therefore, its ability to interfere with iron utilization—a central component of energy metabolism, respiration and DNA synthesis. During microbial respiration, intake of gallium by bacteria disrupts the iron dependent respiratory system leading to the death of the bacteria. While gallium competes with iron for uptake, iron is redox active that allows for the transfer of electrons during respiration, but gallium is redox inactive and thus creates a dead-end to the pathway. Actively proliferating cells rapidly take up gallium, but again as it interferes with cellular iron metabolism like iron's use in DNA synthesis; so the cells cannot reproduce and die by apoptosis. Gallium has low toxic implications because of its poor uptake by normally reproducing cells (as is known from gallium scans). More importantly, gallium does not affect one of the most important iron-bearing proteins—hemoglobin, because of its irreducibility under physiological conditions that prevents it from entering iron (II)-binding molecules such as hemoglobin. Proinflammatory systems in the body increase the iron uptake, so gallium acting as a non-functional iron mimic may alter the immune system and provide anti-inflammatory benefits.

Gallium nitrate (hereinafter “GaN”) has been reported to cause a significant inhibition in growth and to even kill R. equi grown in media. GaN has been used in a mouse model of R. equi pneumonia to decrease R. equi growth in experimentally infected mice. Median tissue concentrations of the pathogen were greater in control mice than in gallium-treated mice. In another study, gallium nitrate was given to pregnant mice to determine the risk of pregnancy related side effects. At 50 mg/kg and below no adverse effects were seen in the fetus but at 100 mg/kg cleft palate and renal hypoplasia were increased. In another report that looked at the effect of GaN on regeneration of bone and cartilage, a dose dependent effect was seen when 300 to 1500 mg/kg of GaN was administered intraperitoneally to Mexican axolotl. Here again, high doses of intraperitoneal GaN were administered.

Virulent R. equi was detected in the feces of all mares suggesting that the mare is an important source of R. equi. Gallium maltolate, when administered to the foal, resulted in an increased gallium serum concentration. But no significant differences were observed in the cumulative incidence of R equi pneumonia between the control and treated groups. There is a need for strategies to control the R. equi infections.

### SUMMARY OF THE INVENTION

One embodiment of the invention is a drug delivery system for reducing the concentration of pathogenic bacteria in feces of a mare. The drug delivery system contains (a) at least one gallium salt in a therapeutically effective amount and in a form that is substantially non-absorbable by a gastrointestinal system of the mare; this at least one specific said gallium salt has an effect of reducing the concentration of R. equi in feces of the mare as compared to feces of the mare prior to receiving the at least one gallium salt; and (b) a pharmaceutically acceptable carrier for delivering the at least one gallium salt to a natural orifice, throat or lungs of the mare. In one embodiment of the invention, the pathogenic bacteria is R. equi.

Another embodiment of the invention is a drug delivery system for preventing respiratory bacterial infection in a foal of a mare, the delivery system contains (a) at least one gallium salt in a therapeutically effective amount and in a form that is substantially non-absorbable by a gastrointestinal system of the mare, this at least one gallium salt has an effect of reducing a concentration of pathogenic bacteria present in feces of the mare as compared to feces of the mare prior to receiving the at least one gallium salt; and (b) a pharmaceutically acceptable carrier for delivering the at least one gallium salt to a natural orifice, throat or lungs of the mare. In one embodiment of the invention, the pathogenic bacteria is R. equi.

Another embodiment of the invention is a method of preventing respiratory bacterial infection in a foal of a mare, the method comprising administering to the mare a drug delivery system including an amount of a therapeutic agent effective to decrease a concentration of R. equi present in a gastrointestinal system of the mare such that feces produced by the mare subjected to the treatment contains a significantly decreased concentration of R. equi as compared to feces of the mare prior to being subjected to the treatment, said therapeutic agent comprising: (a) an amount of at least one gallium salt, said at least one specific said gallium salt being substantially non-absorbable by the gastrointestinal system of the mare and the at least one specific said gallium salt having the effect of decreasing the concentration of R. equi in the mare's feces; and (b) a pharmaceutically acceptable carrier for delivering said at least one gallium salt to the mouth, nose, throat or lungs of the mare, the treatment being administered within a predetermined time range prior to an anticipated date of foaling for the mare.

Another embodiment is a method of preventing infection in a foal of a mare, the method comprising administering to the mare a drug delivery system containing (a) at least one gallium salt in a therapeutically effective amount and in a form that is substantially non-absorbable by a gastrointestinal system of the mare; and (b) a pharmaceutically acceptable carrier for delivering the at least one gallium salt to a natural orifice, throat or lungs of the mare; the drug delivery system has an effect of decreasing the concentration of R. equi in the mare's feces, and the drug delivery system is administered within a predetermined time range prior to an anticipated date of foaling for the mare.

In another embodiment, the predetermined time range is at least one day. In another embodiment, the predetermined time range is at least three days. In another embodiment, the predetermined time range is at least five days. In another embodiment, the gallium salt is selected from the group consisting of gallium nitrate, gallium maltolate, gallium citrate, and gallium phosphate.

### BRIEF DESCRIPTION OF THE DRAWINGS

So that the manner in which the above-recited features, aspects and advantages of the invention, as well as others that will become apparent, are attained and can be understood in detail, more particular description of the invention briefly summarized above can be had by reference to the embodiments thereof that are illustrated in the drawings that form a part of this specification. It is to be noted, however, that the appended drawings illustrate some embodiments of the invention and are, therefore, not to be considered limiting of the invention's scope, for the invention can admit to other equally effective embodiments.

FIG. 1 is a graph showing whole blood concentrations (micromoles/g) of horses in which gallium nitrate was administered daily after pulling blood for two days pre-administration. The horses were treated for five days.

FIG. 2 is a graph showing fecal concentration of virulent R. equi in CFU/g of feces in control mares versus treated at entrance to the study, one week before foaling and one week post foaling. There is a significant reduction in the amount of virulent R. equi in the treated mares over time and versus the control.

FIG. 3 is a graph showing fecal concentration of gallium in ppm of gram of feces in control mares versus treated. There is a significant increase in the fecal concentration of gallium in the treated mares.

### DETAILED DESCRIPTION

Except where stated otherwise, the definitions provided in this document, apply throughout the present specification and claims. These definitions apply regardless of whether a term is used by itself or in combination with other terms.

The term “treating” or “treatment” of a state, disorder, disease or condition as used herein means effecting beneficial or desired results, including clinical results, including but not limited to (1) preventing or delaying the appearance of clinical symptoms of the state, disorder, disease or condition developing in a horse that may be afflicted with or predisposed to the state, disorder, disease or condition but does not yet experience or display clinical or subclinical symptoms of the state, disorder or condition, (2) inhibiting the state, disorder, disease or condition, i.e., arresting or reducing the development of the disease or at least one clinical or subclinical symptom thereof, or (3) relieving the disease, i.e., causing regression of the state, disorder or condition or at least one of its clinical or subclinical symptoms. The benefit to a subject to be treated is either statistically significant or at least perceptible to the veterinarian.

“Effective amount” and “therapeutically effective amount” mean the amount of a compound that, when administered to a horse for treating a state, disorder, disease or condition, is sufficient to effect such treatment. The effective amount or therapeutically effective amount will vary depending on the compound, the disease and its severity, and the age, weight, physical condition and responsiveness of the horse to be treated.

“Delivering” and “administering” means providing a therapeutically effective amount of an active ingredient to a particular location or locations within a host causing a therapeutically effective concentration of the active ingredient at the particular tissue, organ or location in the body. This can be accomplished by any one of the several routes of administration of the active ingredient to the host, including but not limited to local or systemic administration. The therapeutically effective amount may be delivered or administered to a horse as a product comprising specified ingredients in specified amounts, or as a product which results, directly or indirectly, from combination of specified ingredients in specified amounts. The therapeutically effective amount may be delivered or administered to a horse in the form of powders, capsules, syrups, elixirs, tablets, suspensions, solutions or other preparations. The therapeutically effective amount may be delivered or administered to a horse through any of its natural orifices, including, but not limited to, mouth, nose, or anus.

“Pharmaceutically acceptable” means those active agents, salts and esters, and excipients which are, within the scope of sound medical judgment, suitable for use in contact with the tissues of horses without undue toxicity, irritation, allergic response and the like, commensurate with a reasonable benefit/risk ratio, and effective for their intended use.

Examples of gallium salts are gallium bromide, gallium chloride, gallium citrate, gallium fluoride, gallium iodide, gallium maltolate, gallium nitrate, gallium perchlorate, gallium sulfate, gallium phosphate, and gallium citrate. As the absorption of certain gallium salts may be substantial, a gallium salt is selected to be substantially non-absorbed by the mare's gastrointestinal system.

“Substantially non absorbed” means that less than 50% of the drug is absorbed. There are several methods to determine whether a particular gallium salt is substantially non-absorbed by the mare's gastrointestinal system. For example, without limitation, blood levels of gallium may be determined in horses administered either with a gallium salt prepared to be non-absorbable by the gastrointestinal system or with an intravenous injection of a known concentration of gallium. The ratio of the blood concentration levels between the two samples would determine what percent of gallium is absorbed through the gastrointestinal system. In another example, without limitation, measuring the concentration of gallium in the feces compared to the blood concentration would indicate whether a particular gallium salt is substantially non-adsorbed by the mare's gastrointestinal system.

### Example 1

For example, without limitation, in horses fed the same amount, the blood concentration increased from 0.0000231 micromoles/g to 0.0009004 micromoles/g well below what is needed to kill R. equi in the blood, but is a 250% increase in blood concentration over time. Previous in vitro data found anything over 0.050 micromoles/ml killed R. equi in culture, and higher concentrations worked better. Administering oral gallium nitrate to mares daily achieved about 1.5 to 7.1 micromoles of gallium per gram of dry feces. This also led to a reduction in the concentration of R. equi in the feces of the mares.

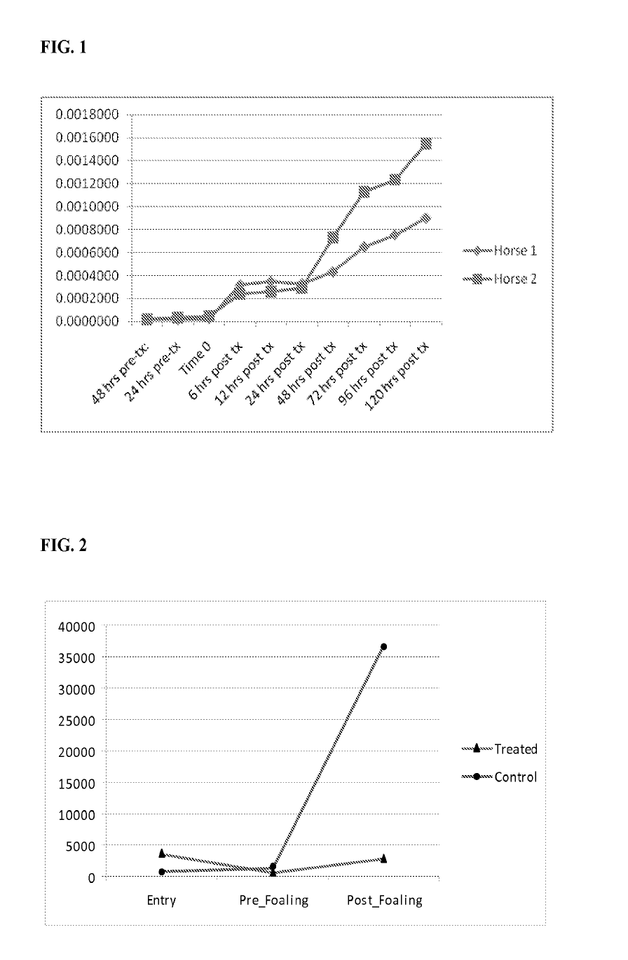

For example, without limitations, two adult horses were given a single dose of gallium nitrate by nasogastric tube at a dose of 9.2 mg/kg at 1% gallium nitrate concentration. Whole blood (WB) and serum samples of Gallium were then measured by spectrometry and demonstrated absorption of the gallium by 6 hours and peaking around 24 hours. Based on tis work, two more horses were dosed with 9.2 mg/kg of daily for 6 days to determine steady state values. As shown in FIG. 1, whole blood concentrations in micromoles/g increased following gallium nitrate administration. There were traces of gallium present in both the red blood cells (“RBC”) and smaller amounts in the serum prior to any medication administration. It appears to concentrate in the RBC and move into the serum. The significance of this is unknown. None of the horses showed any adverse effects from the Gallium treatment. This formulation of gallium has been used safely in over 10,000 horses and there has not been a single claim of any adverse event. It has been used in pregnant mares from conception through foaling with no demonstrable adverse effects.

### Example 2

As part of a non-limiting example of an embodiment of the invention, the effect of oral GaN on the concentrations of virulent R. equi in the feces of the mares was determined. Twenty-one horses were randomly assigned in a paired block design to ensure that 10 mares were part of the treated group and 11 mares were part of the control group. These twenty-one Arabian mares were treated daily with either oral gallium nitrate or placebo. Fecal samples were collected at day 320 of gestation (time 1), the week before foaling (time 2), and the week after foaling (time 3). Airborne concentration of R. equi were measured in the stall within 6 hours post foaling using a microbial air sampling system into which standard (100-mm) culture plates with a media selective for R. equi have been loaded. This sample size of mares was selected on the basis of power calculations using the following assumptions: (a) significance level of P=0.05; (b) statistical power of 90%; (c) a mean of 1×10⁴ colony forming units (cfu) per gram of feces among control mares; (d) a mean of 1×10³ cfu per gram of feces among principal mares; and (e) standard deviations of 1×10⁴ and 1×10³ for the control and principal groups, respectively. The values for fecal concentrations were made on the basis of prior experiences of a collaborator. The total number of R. equi colonies on a plate was determined by morphologic identification; additionally, 10 colonies identified as R. equi each month were tested by PCR to confirm that the isolates are R. equi.

To determine the number of virulent colonies on each plate, a modified colony immunoblot method was used, as previously described (E.g., Grimm M B, Cohen N D, Slovis N M, et al. Evaluation of mares from a Thoroughbred breeding farm as a source of Rhodococcus equi for their foals using quantitative culture and a colony immunoblot assay. Am J Vet Res 2007; 68:63-71). Briefly, this procedure entails applying nitrocellulose membranes to culture plates to transfer bacterial surface proteins, blotting the membranes using a murine monoclonal antibody against the vapA protein, and then using a labeled secondary antibody to detect the presence of vapA-positive (i.e. virulent) colonies. Beginning at day 320 since the last known breeding date for the mare, each mare received a daily dose of 9.2 mg/kg of 1% GaN or an equivalent volume of water given by a dose syringe. Feces was collected on day 320, every following Monday until foaling, the date of foaling, and the Monday after the date of foaling. Fecal samples were collected from a fresh fecal pile that the horse was known to have voided in the stall or by manual evacuation of feces from the rectum. Samples were stored in the refrigerator until shipped chilled and on the day of collection, using overnight courier service.

Concentrations of total R. equi were determined by morphological characteristics. The concentration of virulent R. equi was determined using a modified colony immunoblot method. Concentrations of total and virulent R. equi were compared among mares to examine effects of treatment, time, and treatment by time interaction.

There was a reduction in the concentration of R. equi in the treated group. At time 3 that is the week after foaling, concentrations of virulent R. equi were significantly lower among mares in the treatment group (P<0.05) compared to control. Effects of time depended significantly on the group. For the control group, there were no significant effects with time. For the treatment group, concentrations tended to decrease over time, and concentrations at time 3 were significantly (P<0.05) lower than those at time 1. No other differences among times for concentrations in the treatment group were statistically significant. There was a decrease in the number of mares with positive airborne samples. Five out of 11 control mares had positive air samples. 2 of 10 treated mares had positive samples. Of the foals born to mares with negative airborne samples 6 of 14 were treated for pneumonia. Of the foals with positive samples 5 of 7 were treated. Treatment of mares with oral gallium nitrate significantly reduced the fecal concentrations of virulent R. equi over time, and had an impact on the airborne concentration of R. equi shortly after foaling.

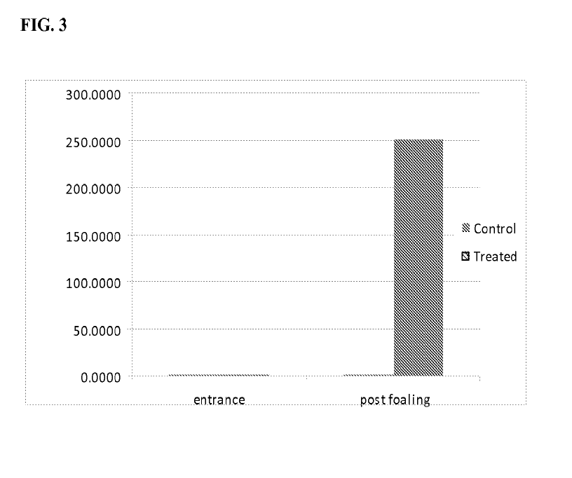

FIG. 2 shows fecal concentration of virulent R. equi in CFU/g of feces in control mares versus treated at entrance to the study, one week before foaling and one week post foaling. There is a significant reduction in the amount of virulent R. equi in the treated mares over time and versus the control. FIG. 3 shows fecal concentration of gallium in ppm of gram of feces in control mares versus treated. There is a significant increase in the fecal concentration of gallium in the treated mares.

### Example 3

As part of a non-limiting example of an embodiment of the invention, virulent R. equi in feces was measured in the feces using the following method. Two milliliters of PBS were added to 1 g of each fecal sample in a conical tube. Thereafter, each sample was vortexed for 10 seconds and centrifuged at 13,000× g for 1 minute. In order to minimize contamination, all pipetting steps were performed in a laminar flow cabinet. Nucleic acid purification from 180 μl of supernatant fluid was performed using an automated nucleic acid extraction system (CAS-1820 X-tractor Gene, Corbett Life Science, Sydney, Australia) according to the manufacturer's recommendations. A real-time TaqMan PCR assay for R. equi has been established and validated. The assay is based on the detection of a specific 75 base-pair long product of the vapA gene of R. equi (GenBank accession number AF116907; oligonucleotides: forward primer CAGCAGTGCGATTCTCAATAGTG, reverse primer CGAAGTCGTCGAGCTGTCATAG, probe CAGAACCGACAATGCCACTGCCTG). Each PCR reaction contained 400 nM of each primer, 80 nM of the TaqMan probe and mastermix (TaqMan Universal PCR Mastermix, Applied Biosystems, Foster City, Calif.) and 1 μL of the gDNA sample in a final volume of 12 μL. The samples were amplified in a combined thermocycler/fluorometer (ABI PRISM 7700 Sequence Detection System, Applied Biosystems, Foster City, Calif.) for 2 min at 50° C., 10 min at 95° C., and then 40 cycles of 15 s at 95° C. and 60 s at 60° C. DNA extraction and amplification efficiency were verified by quantitating the universal bacterial 16S rRNA gene. Absolute quantitation of R. equi target molecules was performed using a standard curve and expressed as R. equi vapA target genes per 1 g of feces. We achieved almost undetectable levels in several treated mares. The highest level in any treated mare was 17,500 CFU/g but most were 1,600 CFU/g or less.

The number of CFU/g of either virulent R. equi in feces was estimated using real-time PCR. Because these samples were collected serially from individual mares, these longitudinal data were analyzed using mixed-effects modeling, with time (number of days since last known breeding date) and treatment group modeled as fixed effects, and individual mare modeled as a random effect. The distribution of the data was examined prior to analysis, and transformed as needed to ensure that data and their variance conform to model assumptions. Analysis was performed using S-PLUS statistical software (Version 8.0; Insightful, Inc.) and a significance level of P<0.05 was sued for significance. The proportion of mares with positive results was compared between groups at baseline (first days of treatment) and at the time of foaling, using chi-squared or Fisher's exact tests. The count of positive samples were determined for each mare, and Poisson regression analysis was used to examine the effects of treatment. Analysis was performed using S-PLUS statistical software (Version 8.0; Insightful, Inc.) and a significance level of P<0.05 was used for significance.

### Example 4

As part of a non-limiting example of an embodiment of the invention, the following method was employed to determine the effect of oral GAN on the airborne concentration of virulent R. equi in foaling stalls shortly after birth. The same 21 mares randomly assigned to GaN treatment or control groups in Example 2 were used in this Example 4. Air samples were collected from the foaling stalls within 12 hours of foaling. Air samples were collected using a commercially available microbiologial air sampling system (M Air T, Millipore, Saint-Quentin-Yveline, France) into which standard (100-mm) culture plates with a media selective for R. equi have been loaded as described in the art. Each sampling entailed aspiration of 1,000 liters of air, which took approximately 10 minutes. The air sampler was placed on the ground to collect air at a height of approximately 4 inches above the stall/floor ground. The day samples were collected and shipped chilled to the Equine Infectious Disease Laboratory at Texas A&M University for enumeration of the total number of R. equi colonies and the number of virulent R. equi colonies on each plate. The total number of R. equi colonies on a plate were determined by morphologic identification; additionally, 10 colonies identified as R. equi each month were tested by PCR to confirm that the isolates were R. equi. To determine the number of virulent colonies on each plate, a modified colony immunoblot method was used, as previously described. (E.g., G. Muscatello and G. Browning, Identification and differentiation of avirulent and virulent Rhodococcus equi using selective media and colony blotting DNA hybridization to determine their concentrations in the environment. Veterinary Microbiology, 100 (2004): 121-127). This procedure entailed applying nitrocellulose membranes to culture plates to transfer bacterial surface proteins, blotting the membranes using a murine monoclonal antibody against the vapA protein, and then using a labeled secondary antibody to detect the presence of vapA-positive (i.e., virulent) colonies. The number of total and virulent R. equi counted on each plate represented the airborne concentrations (cfu/1,000 liters of air). Fecal and air samples were analyzed for concentration of virulent R. equi.

The outcome (dependent variable) for analysis of the airborne sample analyses were either the concentration of total R. equi or virulent R. equi in air. Zero-inflated negative binomial regression method was used to model the association between airborne concentration of R. equi and treatment group. Results indicate that treatment of mares with oral gallium nitrate significantly reduced fecal concentrations of virulent R. equi but had no statistically significant impact on airborne concentrations. Other likely factors, possibly related to previous stall occupants and farm environments, also contribute to the airborne concentration in stalls. Reducing the R. equi burden in the environment of a foal will be important in decreasing incidence of disease.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description to the invention. Changes may be made to the elements described herein without departing from the spirit and scope of the invention as described in the following claims. In addition, it is to be understood that features described herein independently may, in certain embodiments, be combined.

What is claimed is:

1. A drug delivery system for reducing the concentration of pathogenic bacteria in feces of a mare, the drug delivery system comprising:
at least one gallium salt in a therapeutically effective amount and in a form that is substantially non-absorbable by a gastrointestinal system of the mare;
the at least one specific said gallium salt has an effect of reducing the concentration of pathogenic bacteria in feces of the mare as compared to feces of the mare prior to receiving the at least one gallium salt; and
a pharmaceutically acceptable carrier for delivering the at least one gallium salt to a natural orifice, throat or lungs of the mare.

2. The drug delivery system of claim 1, wherein the pathogenic bacteria is Rhodococcus equi.

3. The drug delivery system of claim 1, wherein the gallium salt is selected from a group consisting of gallium bromide, gallium chloride, gallium citrate, gallium fluoride, gallium iodide, gallium maltolate, gallium nitrate, gallium perchlorate, gallium sulfate, gallium phosphate, and gallium citrate.

4. A drug delivery system for preventing respiratory bacterial infection in a foal of a mare, the delivery system comprising:
at least one gallium salt in a therapeutically effective amount and in a form that is substantially non-absorbable by a gastrointestinal system of the mare,
the at least one gallium salt has an effect of reducing a concentration of pathogenic bacteria present in feces of the mare as compared to feces of the mare prior to receiving the at least one gallium salt; and
a pharmaceutically acceptable carrier for delivering the at least one gallium salt to a natural orifice, throat or lungs of the mare.

5. The drug delivery system of claim 4, wherein the pathogenic bacteria is Rhodococcus equi.

6. The drug delivery system of claim 4, wherein the gallium salt is selected from a group consisting of gallium bromide, gallium chloride, gallium citrate, gallium fluoride, gallium iodide, gallium maltolate, gallium nitrate, gallium perchlorate, gallium sulfate, gallium phosphate, and gallium citrate.

7. A method of preventing infection in a foal of a mare, the method comprising
administering to the mare a drug delivery system comprising
at least one gallium salt in a therapeutically effective amount and in a form that is substantially non-absorbable by a gastrointestinal system of the mare; and
a pharmaceutically acceptable carrier for delivering the at least one gallium salt to a natural orifice, throat or lungs of the mare, the drug delivery system has an effect of decreasing the concentration of R. equi in the mare's feces, and
the drug delivery system is administered within a predetermined time range prior to an anticipated date of foaling for the mare.

8. The method of claim 7, wherein the predetermined time range is at least one day.

9. The method of claim 7, wherein the predetermined time range is at least three days.

10. The method of claim 7, wherein the predetermined time range is at least five days.

11. The method of claim 7, wherein the gallium salt is selected from a group consisting of gallium bromide, gallium chloride, gallium citrate, gallium fluoride, gallium iodide, gallium maltolate, gallium nitrate, gallium perchlorate, gallium sulfate, gallium phosphate, and gallium citrate.
